(19) United States
(12) Patent Application Publication
(10) Pub. No.: US 2014/0296612 A1
(43) Pub. Date: Oct. 2, 2014

(54) Brachytherapy Assist Device

(71) Applicant: David Schwartz, Brookville, NY (US)

(72) Inventor: David Schwartz, Brookville, NY (US)

(21) Appl. No.: 13/941,264

(22) Filed: Jul. 12, 2013

Related U.S. Application Data
(63) Continuation-in-part of application No. 13/873,475, filed on Apr. 30, 2013.
(60) Provisional application No. 61/805,455, filed on Mar. 26, 2013.

(51) Int. Cl. A61N 5/10

(52) U.S. Cl. CPC A61N 5/1007; A61N 2005/1094
USPC 600/8; 600/7

(57) ABSTRACT

A lightweight, portable, interchangeable and effective overlay plate that will increase the efficiency of brachytherapy radiation treatment and reduce the duration of the radiation exposure during the radioactive seed insertion and positioning in brachytherapy. The overlay plate has a limited number of apertures specific to the treatment of a particular patient and is attached to the matrix used to position and guide the needles with the radioactive seeds. The apertures of the overlay plate may further be truncated-cone-shaped, with the narrow ends aligned with corresponding apertures in the matrix and the flared ends facing the user. The overlay plate minimizes the radiation exposure as much as reasonably possible because any amount of radiation exposure carries a risk to the exposed individual, and the overlay plate may also be manufactured from radiation-protective material to further minimize the radiation exposure.

### CROSS REFERENCE OF RELATED APPLICATION

This patent application is a continuation-in-part application of nonprovisional patent application Ser. No. 13/873,475 filed on Apr. 30, 2013, which was a nonprovisional application of provisional patent application Ser. No. 61/805,455 filed on Mar. 26, 2013.

### BACKGROUND

This invention relates to device that speeds up the process of internal radiotherapy treatment and makes it more efficient, as well as minimizes the duration of the radiation exposure of the treating physician, who is typically a radiation oncologist or a surgeon, during the cancer treatments of patients. The device is preferably a substantially flat or planar plate with a limited number of apertures specific to the prescribed treatment of a patient. The device of the present invention may also have radiation absorptive and/or protective properties and may serve as a radiation protective shield in addition to making the treatment process more efficient.

Brachytherapy is short-distance internal radiotherapy, in which small radioactive seeds or pellets are implanted into tumors of cancerous tissue and kill cancerous cells. Brachytherapy is often an alternative to the radically-invasive surgical tumor removal, or external beam radiation. Brachytherapy is a more localized and targeted treatment, and it has been successfully used to treat various cancers, including prostate cancer that is used as an example to describe the present invention.

Prostate cancer is the second leading cause of cancer death in men, and this cancer accounts for approximately a third of all male cancers. Early diagnosis and treatment of the disease significantly improves survival chances and minimizes harmful side effects from treatment. Prostate biopsies are a common way to diagnose the cancer in a male patient's prostate. The prostate biopsies are typically performed through the rectum and are called in the medical field “a transrectal biopsy.” An alternative way to perform prostate biopsies is transperineally, or through the perineum, which is the tissue between the testicles and the anus of a male patient.

In prostate brachytherapy, patients are placed in the lithotomy position, where the patient is supine on his back, and the patient's legs are elevated and/or bent. The lithotomy position allows for unobstructed access to the perineum.

With an ultrasound probe in the rectum looking at the prostate just anterior to the rectum, needles are placed through the skin of the perineum into the prostate. A template grid that corresponds to an associated grid on the ultrasound screen is utilized to guide the needles. Other guiding methods, such as x-ray and fluoroscopy, have also been used. The template grid is typically a square matrix with evenly-spaced apertures for inserting the needles, as illustrated in FIG. 3, and each of the apertures is associated with a horizontal and a vertical coordinates.

In prostate brachytherapy, which uses radiation sources placed in or near the area to be treated, small radioactive seeds or pellets are implanted into cancerous tissue in the prostate through needles. The needles are guided by the matrix through the perineum and into the prostate to properly position and implant the seeds. This is also called permanent brachytherapy or seed implantation. The template grid and the insertion of the needles guided by ultrasound allow for precise positioning of the radioactive seeds inside the cancerous tumor tissue.

When implanted, the radioactive seeds remain lodged in the cancerous tissue and continue delivering a localized dose of radiation directly to the cancerous tissue.

However, the radioactive seeds inserted into and positioned inside the prostate expose treating physicians to radiation, which, even in small amounts, is highly undesirable if the exposure is prolonged from patient to patient. The radioactive seeds typically used are Iodine-125, Palladium-103 and Cesium-131, with the radiation emitted from 04 to 2.5 u per seed, with average energy of 21 keV for PD-103, 29.5 keV for 1-125, and 30.4 keV for Cs-131. However, dozens or sometimes over a hundred seeds are implanted into any given patient, and the radiation exposure is multiplied by the number of patients treated every day.

### DESCRIPTION OF PRIOR ART

The current state of the art involves using a simple square matrix, which may have well over 100 apertures. The square matrix illustrated in FIG. 3 has 169 apertures in a square grid, 13 horizontal apertures by 13 vertical apertures. The square matrix is relatively small. It takes the treating physician time to identify and select the proper apertures to insert the needles through, and that makes the treatment procedure take more time and increases the duration of the radiation exposure to the treating physician. The use of the small square matrix with so many apertures also requires unnecessary levels of manual dexterity and eyesight from the physician.

In addition, the square matrix offers little or no protection against the radiation emitted by the radioactive seeds. The insertion and positioning of the radioactive seeds and the removal of the needles is performed without any portable shielding that would protect the physician during the procedure.

What is needed is a lightweight, portable overlay for the matrix that will have a limited number of apertures, specific to the individual patient and the treatment prescribed to the patient. The overlay is aligned with the square matrix and the overlay simply covers all the unnecessary apertures of the square matrix, leaving only the apertures necessary for the treatment of the specific patient.

As an added feature, the overlay may serve as a shield that will protect the physician from radiation during the procedure. Any radiation exposure, no matter how small, carries some risk to the exposed individual. There is a known concept of “ALARA”, which stands for “As Low As Reasonably Achievable” and is related to minimizing the exposure associated with use of radiation or radioactive materials, especially exposure that is unnecessary and could be avoided. ALARA utilizes shielding, whenever possible, as one of the basic protective measures used to minimize the radiation exposure, together with reducing the time of the exposure, and maximizing the distance from the radiation source.

### SUMMARY

This invention meets the current need for a brachytherapy assist device. A novel overlay device is provided. The preferred embodiment of the overlay device is a substantially flat plate, with a limited number of apertures, that attaches to the matrix and is aligned with the matrix so as to leave only the apertures to be used during the specific treatment procedure open and accessible. The overlay follows and fits with the ALARA concept, minimizing the duration of the radiation exposure associated with the handling and use of the radioactive seeds. The overlay may also be manufactured from radiation protective material so as to shield the user from radiation, further increasing the safety of the procedure to the treating physician.

The overlay is preferably removably attached to the matrix for ease of cleaning and replacement, and because a particular overlay with its specific limited number of apertures may only be used with specific patients. A set of the overlays, having different apertures, is preferably used with any standard matrix, simplifying and speeding up the treatment procedure. Each overlay is essentially a template for the brachytherapy treatment, having only the apertures required for that treatment. The preferred method of attachment of the overlay to the matrix uses two spring clips as illustrated in FIG. 1.

### BRIEF DESCRIPTION OF THE DRAWINGS

These features, aspects and advantages of the novel overlay device and a method of use thereof will become further understood with reference to the following description and accompanying drawings where

FIG. 1 is an isometric view of the matrix and the attached overlay device of the present invention;

FIG. 2 is a front down view of the matrix and the attached overlay device of the present invention illustrated in FIG. 1; and

FIG. 3 is a close-up view of the matrix (the template grid).

### DESCRIPTION

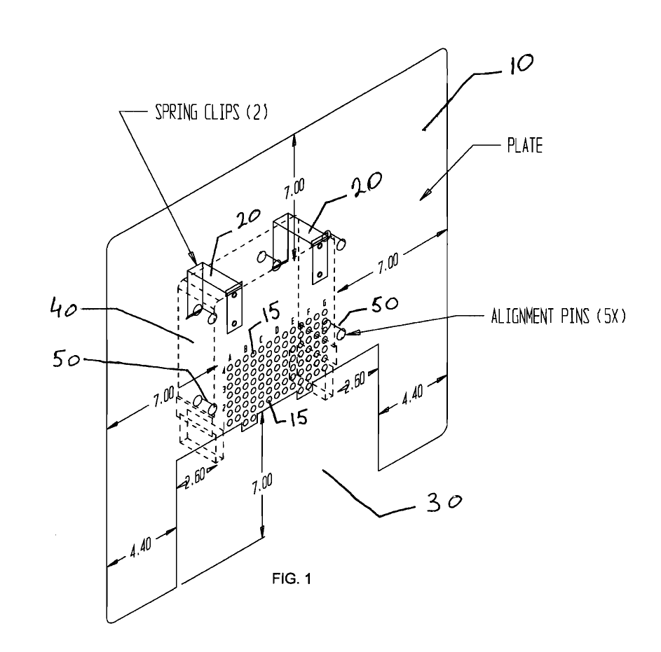

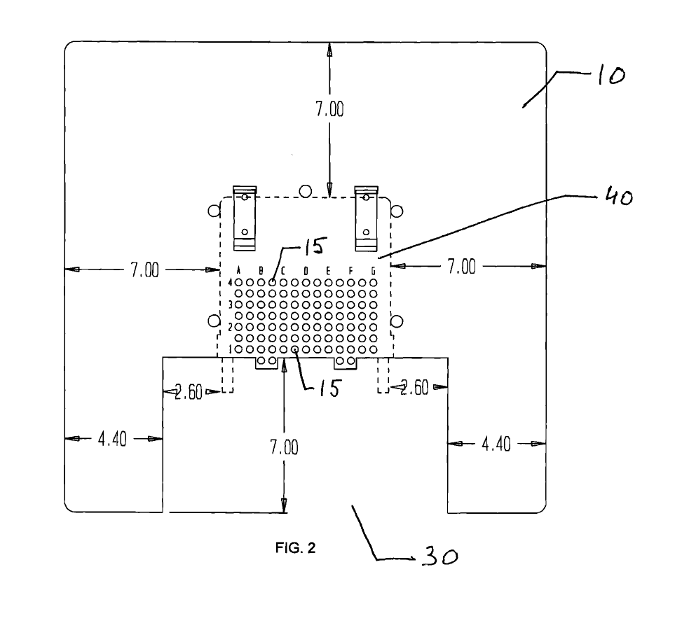

The present invention is directed to an effective, lightweight, portable overlay device and a method of use thereof. A preferred embodiment is shown in FIGS. 1 and 2. With reference to the drawings, and particularly FIGS. 1 and 2, one embodiment of the invention comprises a substantially flat overlay plate 10 removably attached to the matrix 40 using two spring clips 20 fastened to the overlay plate, which clips 20 snap onto the matrix 40 over the top surface of the matrix 40.

However, other types and numbers of clips or brackets may be used to attach the overlay plate 10 to the matrix 40 and to hold it in place, such as C-shaped brackets for example. The clips or brackets may snap in or otherwise securely fasten the overlay plate 10 to the matrix 40. It is also possible to use railed attachment methods for a stable coupling of the overlay plate 10 and the matrix 40.

The overlay plate 10 is preferably portable, capable of being lifted and manipulated without strain by an average user. The size of the overlay plate 10 may be varied based on the need dictated by the application of the radioactive seeds and a variety of other factors, such as whether the overlay plate 10 also serves as a radiation protective shield. Therefore, it is preferable to have an overlay plate 10 that is easily removable, i.e., by detaching or disengaging the clips 20 (or clamps, brackets or other attachment means), so that a number of interchangeable overlay plates of different size, material and thickness may be used with the matrix 40.

The overlay plate 10, when attached to the matrix 40 and is in use, is preferably positioned between the matrix 40 and the treating physician. The overlay plate 10 is preferably the same size (i.e., has the same horizontal and vertical dimensions and covers the same area) as the matrix 40, but the overlay plate 10 may also extend beyond the edges of the matrix 40 if the overlay plate 10 is manufactured from radiation-protective material and serves as a radiation protective shield. In the shown embodiment in FIGS. 1 and 2, where the overlay plate 10 is also used as a protective shield, there is a cutout 30 in the overlay plate 10 below the bottom edge of the matrix 40. The cutout 30 is necessary to avoid other equipment used in the procedure.

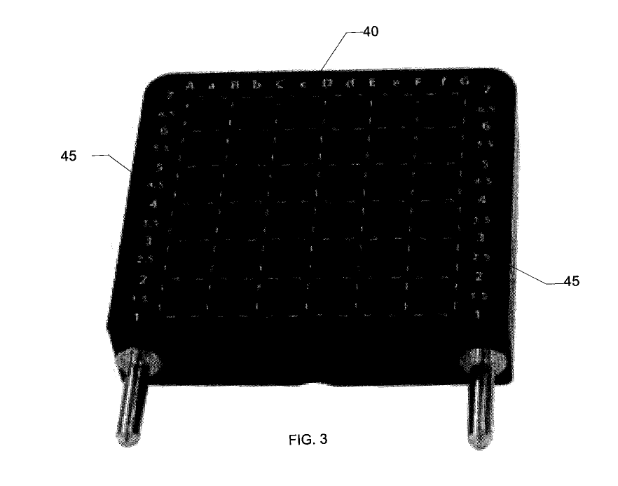

When the overlay plate 10 covers the entire matrix 40, which is the preferred configuration, the overlay plate 10 has apertures 15 corresponding to the apertures 45 on the matrix 40, illustrated in FIG. 3, to allow the needles with the radioactive seeds to pass through the overlay plate 10 and the matrix 40. There are preferably fewer apertures 15 than apertures 45 on the overlay plate 10, the number of apertures 15 being specific to a particular patient and the prescribed treatment procedure. Having a limited number of apertures 15 in the overlay plate 10 greatly increases the efficiency of the brachytherapy treatment because the treating physician does not have to spend as much time on identifying the proper apertures in the matrix 40 out of 169 apertures.

The apertures 15 may further be flared towards the user side of the overlay plate 10. The flared, or truncated-cone-shaped, apertures 15 have the narrow ends of the truncated cones aligned with the corresponding apertures 45 on the matrix 40, and the flared ends facing the user. Having cone-shaped apertures 15 helps the user find and insert needles into correct apertures much faster, thereby further increasing the efficiency of the procedure.

The increased efficiency of the treatment procedure also reduces the duration of the radiation exposure for the treating physician. When in use, the overlay plate 10 of the present invention may also shield the user during the brachytherapy procedure if it is manufactured from radiation absorbing material. More specifically, the overlay plate 10 of the present invention may protect the user from radiation during the insertion and positioning of the radioactive seeds, while the user inserts the needles with the radioactive seeds through the apertures 15 of the overlay plate 10 and the apertures 45 of the matrix 40, punctures the skin in the perineum area with the needles and implants the seeds into the prostate, where the radioactive seeds are positioned using the vertical and horizontal coordinates associated with the apertures 45 on the matrix 40, ultrasound imaging or similar guidance, and the length of the needles.

To facilitate easier attachment of the overlay plate 10 to the matrix 40, as well as the proper, symmetric alignment of the overlay plate 10 with the matrix 40, the overlay plate 10 preferably has alignment pins 50 as illustrated in FIG. 1, which are preferably structures attached to the posterior surface of the overlay plate 10 that hold the matrix 40, also known as the template or grid, in place.

The preferred embodiment uses alignment pins 50 attached to the posterior of the overlay plate 10 only, and, as illustrated in FIGS. 1 and 2, there are five alignment pins 50 in the preferred embodiment that are positioned around the side edges and the top edge of the matrix 40. The alignment pins 50 are essentially pegs that may be formed with the overlay plate 10 or formed separately and affixed to the posterior of the overlay plate 10 (for example riveted). The placement of the alignment pins 50 allows for the proper alignment of the overlay plate 10 with the matrix 40, so as to ensure the alignment of the apertures 45 in the matrix and the apertures 15 in the overlay plate 10. This attachment method and location of the alignment pins 50 also allow for the use of the overlay plate 10 of the present invention with any standard size matrix.

The limited number of apertures 15 in the overlay plate 10 may have associated vertical and horizontal coordinates as well as the matrix 40. However, provided the proper alignment of the overlay plate 10 and the matrix 40 is achieved, the overlay plate 10 does not have to include coordinates for its apertures 15.

The overlay plate 10 is preferably made from clear plastic because that still permits the treating physician to observe the entire square matrix 40 and use it as a secondary reference for the insertion of the needles and positioning of the seeds. However, it is not necessary to manufacture the overlay plate 10 from a see-through material. Various other materials of different opacity and with radiation shielding properties may be used for the overlay plate 10, such as stainless steel, leaded glass or acrylic glass used to protect against X-Ray exposure, as well as numerous other materials and compounds. Other embodiments of the present invention may use an acrylic overlay plate 10 due to its light weight and resistance to shattering. The corners and the corner edges of the overlay plate 10 are preferably rounded and/or smoothed so as to avoid any cut or other injury to the patient or the user.

The overlay plate 10 and method of use thereof of the present invention have other possible uses. Although the present invention has been described in terms of use for prostate cancer treatment, brachytherapy has been successfully used for breast, cervical, lung, skin, head, and neck cancers, and the present invention can be applied to all of these treatments. Likewise, it may be used for contact brachytherapy where the radiation sources are placed outside of, but next to, the targeted tumor, as well as internal brachytherapy. The present invention is also useful for taking prostate biopsies, including biopsies in the sit-up position.

The above description of the disclosed preferred embodiments is provided to enable any person skilled in the art to make or use the invention. Various modifications to these embodiments will be readily apparent to those skilled in the art, and the principles described herein can be applied to other embodiments without departing from the spirit or scope of the invention and the subject matter of the present invention, which is broadly contemplated by the Applicant. The scope of the present invention fully encompasses other embodiments that may be or become obvious to those skilled in the art.

1. A novel overlay plate for increasing the efficiency of a prescribed brachytherapy treatment of a patient utilizing a matrix and decreasing the duration of radiation exposure to the user, the overlay plate comprising:
(a) a substantially flat plate attached to the matrix, said substantially flat plate having a limited number of apertures corresponding to the prescribed brachytherapy treatment of the patient.

2. The novel overlay plate of claim 1, wherein the limited number of apertures of the substantially flat plate cooperate with a plurality of apertures in the matrix suitable for brachytherapy needles, each of the plurality of apertures having associated vertical and horizontal coordinates.

3. The novel overlay plate of claim 1, wherein the substantially flat plate is removably attached to the matrix.

4. The novel overlay plate of claim 3, wherein the substantially flat protective plate is removably attached to the matrix by at least one spring clip.

5. The novel overlay plate of claim 4, wherein the at least one spring clip couples with a top surface of the matrix to removably attach the substantially flat plate to the matrix.

6. The novel overlay plate of claim 2, wherein each of the limited number of apertures of the substantially flat plate is formed as a truncated cone having a narrow end and a flared end.

7. The novel overlay plate of claim 6, wherein the narrow end of each of the limited number of apertures is aligned with a corresponding aperture of the plurality of apertures in the matrix, and wherein the flared end faces the user.

8. The novel overlay plate of claim 1, wherein the substantially flat plate includes a plurality of alignment pins to facilitate the alignment of the substantially flat plate and the matrix.

9. The novel overlay plate of claim 8, wherein each of the plurality of alignment pins is a structure attached to a surface of the substantially flat plate.

10. The novel overlay plate of claim 1, wherein the substantially flat plate is portable, capable of being lifted and manipulated by an average user.

11. The novel overlay plate of claim 1, wherein corners of the substantially flat plate are rounded so as to avoid injury to the patient or the user.

12. The novel overlay plate of claim 1, wherein edges of the substantially flat plate are smoothed so as to avoid injury to the patient or the user.

13. A novel overlay plate for increasing the efficiency of a prescribed brachytherapy treatment of a patient utilizing a matrix and decreasing the duration of radiation exposure to the user, the overlay plate comprising:
(a) a substantially flat plate having at least one fastener for removably attaching the substantially flat plate to the matrix, said substantially flat plate having a limited number of apertures corresponding to the prescribed brachytherapy treatment of the patient.

14. The novel overlay plate of claim 13, wherein the at least one fastener is selected from the group consisting of clips, spring clips, clamps, brackets, magnets, Velcro, reciprocating rails and combination thereof.

15. A novel method of increasing the efficiency of a prescribed brachytherapy treatment of a patient utilizing a matrix and decreasing the duration of radiation exposure to the user during brachytherapy treatment of a patient, comprising:
(a) attaching a substantially flat plate to a matrix, said substantially flat plate having a limited number of apertures corresponding to the prescribed brachytherapy treatment of the patient and aligning the substantially flat plate with the matrix so that the limited number of apertures is aligned with a plurality of cooperating apertures in the matrix suitable for brachytherapy needles, wherein each of the plurality of cooperating apertures is associated with a vertical coordinate and a horizontal coordinate;
(b) positioning the substantially flat plate between the matrix and the user, wherein the matrix is positioned between the patient and the substantially flat plate;
(c) inserting at least one brachytherapy needle having at least one radioactive seed therein through at least one of the limited number of apertures and at least one corresponding aperture of the plurality of cooperating apertures;
(d) puncturing skin of the patient near a region of cancerous tissue with the at least one brachytherapy needle; and
(e) positioning the at least one radioactive seed inside the cancerous tissue.

16. The method of claim 15, wherein the positioning of the radioactive seeds is performed using predetermined vertical and horizontal coordinates associated with the at least one corresponding aperture of the plurality of cooperating apertures on the matrix cooperating with the at least one of the limited number of apertures of the substantially flat plate.

17. The method of claim 15, wherein the positioning of the at least one radioactive seed is performed using ultrasound imaging.

18. The method of claim 15, wherein the positioning of the at least one radioactive seed is performed using the length of the at least one brachytherapy needle.

19. The method of claim 15, wherein each of the limited number of apertures has a narrow end and a flared end, each said narrow end being aligned with a corresponding aperture of the plurality of cooperating apertures in the matrix and each flared end facing the user after the step of attaching the substantially flat plate to the matrix.
